(19) United States
(12) Patent Application Publication
(10) Pub. No.: US 2008/0221150 A1
(43) Pub. Date: Sep. 11, 2008

(54) Prevention of Neurodegeneration by Macrolide Antibiotics

(76) Inventors: John J. Shacka, Birmingham, AL (US); Kevin A. Roth, Birmingham, AL (US)

Correspondence Address: BRADLEY ARANT ROSE & WHITE, LLP;INTELLECTUAL PROPERTY DEPARTMENT-NWJ, 1819 FIFTH AVENUE NORTH, BIRMINGHAM, AL, 35203-2104, US

(21) Appl. No.: 12/063,715

(22) PCT Filed: Aug. 14, 2006
(86) PCT No.: PCT/US2006/031619

§ 371 Date: Feb. 13, 2008

Related U.S. Application Data
(60) Provisional application No. 60/707,585, filed on Aug. 13, 2005.

(51) Int. Cl. A61K 31/335; A61K 31/47; A61K 31/4375; A61P 25/00

(52) U.S. Cl. 514/300; 514/450; 514/313

(57) ABSTRACT

The present disclosure describes the use of macrolide antibiotics to prevent neurodegeneration and to treat or prevent disease and conditions which involve neurodegeneration. While not being limited to a specific mechanism of action, in one embodiment, the macrolide antibiotics inhibit neurodegeneration caused by lysosomal and/or mitochondrial dysfunction. The present disclosure contemplates the use of any macrolide antibiotic and pharmaceutically acceptable derivatives thereof. In a specific embodiment, the macrolide antibiotics include bafilomycin A1, bafilomycin B1 and concanamycin.

This application claims priority to and benefit of U.S. Provisional application No. 60/707,585, filed Aug. 13, 2005.

### STATEMENT AS TO FEDERAL FUNDING

The work described herein was supported by Federal Research Grant NIH-NINDS RO1-35107. As such, the Federal Government has certain rights in this invention.

### FIELD OF THE DISCLOSURE

The present disclosure is directed to methods for the treatment and/or prevention of neurodegeneration and diseases and conditions characterized by neurodegeneration. Such methods may be used to treat a subject suffering from a disease or condition characterized by neurodegeneration and/or to prevent the occurrence of such disease or condition in an at risk subject. The present disclosure also relates to pharmaceutical compositions useful in such methods.

### BACKGROUND

Neurodegeneration is a commonly used term the meaning of which is believed to be universally understood. However, a precise definition for neurodegeneration is difficult to formulate. Often, neurodegeneration is incompletely defined in the most comprehensive dictionaries. Etymologically, the word is composed of the prefix “neuro-,” which designates nerve cells (i.e., neurons), and “degeneration,” which refers to, in the case of tissues or organs, a process of losing structure or function, which process may be reversible with treatment.

In practice, neurodegenerative diseases and conditions represent a large group of neurological disorders with heterogeneous clinical and pathological expressions affecting specific subsets of neurons in specific functional anatomic systems. These disorders may be observed early in life (early onset disorders) or late in life (age-related disorders) that arise for unknown reasons and progress in a relentless manner. The most consistent risk factor for developing a neurodegenerative disease or condition, such as Alzheimer's disease or Parkinson's disease, is increasing age.

Over the past century, the growth rate of the population aged 65 and beyond in industrialized countries has far exceeded that of the population as a whole. Thus, it can be anticipated that, over the next generations, the proportion of elderly citizens will double, and, with this, possibly the proportion of persons suffering from some kind of neurodegenerative disease or condition. This prediction is at the center of growing concerns in the medical community due to the increasing magnitude of emotional, physical, and financial burdens on patients, caregivers, and society that are related to these disabling illnesses. Compounding the problem is the fact that while, to date, several approved drugs do, to some extent, alleviate symptoms of several neurodegenerative diseases and conditions, their chronic use is often associated with debilitating side effects, and none seem to stop the progression of the degenerative process. In keeping with this, the development of effective preventive or protective therapies has been impeded by the limitations of our knowledge of the causes and the mechanisms of neurodegeneration. Therefore, there exists a need in the art for new compounds for treatment of neurodegenerative diseases and conditions and methods of treatment using such compounds. Furthermore, methods for identifying suitable compounds for the treatment and/or prevention of neurodegenerative disorders are also needed.

The present disclosure discloses the use of macrolide antibiotics in the treatment and/or prevention of neurodegeneration and diseases and conditions characterized by neurodegeneration.

The present disclosure also discloses methods of treatment and/or prevention using such macrolide antibiotics. Furthermore, the present disclosure also relates to pharmaceutical compositions useful in such methods. The present disclosure also provides methods for identifying other compounds that may be effective in treating and/or preventing neurodegenerative diseases and conditions.

### BRIEF DESCRIPTION OF THE FIGURES

FIGS. 1A-1D shows that chloroquine decreases cell viability and increases caspase-3-like activity in cerebellar granule neurons (CGNs). CGNs were isolated and cultured in vitro for 4 days as described in the Methods section and viability (FIG. 1A) expressed as % control (CTL) and fractional caspase-3-like activity (a marker of apoptosis) (FIG. 1B) were measured 24 hours following treatment with chloroquine (5-40 μM) as described in the Methods section. Viability (FIG. 1C) and caspase-3-like activity (FIG. 1D) were also measured for up to 48 hours following treatment with 20 μM chloroquine as described in the Methods section. Three independent experiments of at least three replicates represent each time point and concentration tested. Significant effects of treatment and time were assessed via one-factor ANOVA with Bonferonni's post hoc test. For FIGS. 1A and 1B, p<0.05 (*compared to CTL; ^{†}compared to 5 μM chloroquine; ^{‡}compared to 10 μM chloroquine; ^{#}compared to 20 μM chloroquine). For FIGS. 1C and 1D, p<0.05 (*compared to 0 hour; ^{†}compared to 8 hours; ^{‡}compared to 16 hours; ^{#}compared to 24 hours).

FIGS. 2A-2E show that chloroquine induces biochemical markers of apoptosis (caspase-3) and autophagic stress (LC3-II) in CGNs. CGNs were isolated and cultured in vitro for 4 days as described in the Methods section. A representative western blot (FIG. 2A) shows determination of cleaved caspase-3 and processing of LC3 in non-treated (NT) samples at 0 hours and in control (CTL) or chloroquine (20 μM; CQ)-treated lysates from 4-48 hours. At least three independent experiments were used to assess levels cleaved caspase-3 (a marker of apoptosis) (FIG. 2B), LC3-I (FIG. 2C) and LC3-II (a marker of autophagic stress) (FIG. 2D), expressed relative to levels of β-III-tubulin, and the ratio of LC3-II/I (FIG. 2E).

FIGS. 3A and 3B show that Bafilomycin A1 decreases viability and increases caspase-3-like activity in a concentration dependent manner in CGNs. CGNs were isolated and cultured in vitro for 4 days as described in the Methods section. Viability (FIG. 3A) and fractional caspase-3-like activity (a marker of apoptosis) (FIG. 3B) were measured 24 hours following the addition of Bafilomycin A1 (BafA1) (0-100 nM). Three independent experiments of at least three replicates represent each time point and concentration tested. Significant effects of treatment were assessed via one-factor ANOVA with Bonferonni's post hoc test; p<0.05 (*compared to 0, 0.1 and 0.3 nM Bafilomycin A1; ^{†}compared to 10 nM Bafilomycin A1).

FIGS. 4A-4D show that Bafilomycin A1 modulates markers of apoptosis and autophagic stress in a dose dependent manner in CGNs. CGNs were isolated and cultured in vitro for 4 days as described in the Methods section. A representative western blot (FIG. 4A) shows the determination of cleaved caspase-3 and processing of LC3 in lysates in control cells (CTL) and 24 hours addition of various concentrations of Bafilomycin A1 (BafA1). At least three independent experiments were used to assess levels of cleaved caspase-3 (a marker of apoptosis) (FIG. 4B), LC3-I and LC3-II (a marker of autophagic stress) (FIG. 4C) expressed relative to levels of β-III-tubulin, and the ratio of LC3-II/LC3-I (FIG. 4D).

FIGS. 5A-5C show that Bafilomycin A1 attenuates the chloroquine-induced, but not staurosporine-induced, decrease in viability and increase in caspase-3-like activity in a concentration dependent manner in CGNs. CGNs were isolated and cultured in vitro for 4 days as described in the Methods section. Viability (FIG. 5A) and fractional caspase-3-like activity (FIG. 5B) were measured 24 hours following the co-addition of 20 μM chloroquine (CQ) and Bafilomycin A1 (BafA1) (0-100 mM). Viability (FIG. 5C) was measured 24 hours following the co-addition of Bafilomycin A1 (BafA1) (0-10 nM) and staurosporine (0.1 μM) (STS). Bafilomycin A1 attenuated the decrease in viability and caspase-3 activation (a marker of apoptosis) induced by chloroquine, but did not prevent the decrease in viability following 24 hours treatment with staurosporine. Three independent experiments of at least three replicates represent each concentration tested. For FIGS. 5A and 5B, significant effects of Bafilomycin A1 (in the presence or absence of CQ) were assessed via one-factor ANOVA with Bonferonni's post hoc test; p<0.05 (*compared to CTL; ^{†}compared to CQ; ^{‡}compared to 0.1 nM Bafilomycin A1; ^{#}compared to 0.3 nM Bafilomycin A1; ^{§}compared to 1 nM Bafilomycin A1). For FIG. 5C, significant effects of staurosporine (in the presence or absence of Bafilomycin A1) were assessed via two-factor ANOVA and concentration-dependent differences of Bafilomycin A1 were assessed via one-factor ANOVA with Bonferonni's post hoc test; p<0.05 (*compared to each concentration of Bafilomycin A1 without staurosporine; ^{†}compared to 0 and 1 nM Bafilomycin A1).

FIGS. 6A-6D show that Bafilomycin A1 attenuates chloroquine-induced cleavage of caspase-3 and the ratio of LC3-II/LC3-I in a dose dependent manner in CGNs. CGNs were isolated and cultured in vitro for 4 days as described in the Methods section. A representative western blot (FIG. 6A) shows cleaved caspase-3 and processing of LC3 in samples treated for 24 hours with Bafilomycin A1 (BafA1) (0.3-10 nM) in the presence or absence of 20 μM chloroquine (CQ). At least three independent experiments were used to assess levels of cleaved caspase-3 (a marker of apoptosis) (FIG. 6B), LC3-I and LC3-II (a marker of autophagic stress) (FIG. 6C) expressed relative to levels of β-III-tubulin, and the ratio of LC3-II to LC3-I (FIG. 6D).

FIGS. 7A-7F show the effects of chloroquine and Bafilomycin A1 on the labeling of acidic vesicles in CGNs. CGNs were isolated and cultured in vitro for 4 days as described in the Methods section. CGNs were treated for 24 hours as follows: (FIG. 7A) Control (CTL); (FIG. 7B) 1 nM Bafilomycin A1 (BafA1); (FIG. 7C) 10 nM Bafilomycin A1 (BafA1); (FIG. 7D) 20 μM chloroquine (CQ); (FIG. 7E) 20 μM chloroquine (CQ)+1 nM Bafilomycin A1 (BafA1); (FIG. 7F) 20 μM chloroquine (CQ)+10 nM Bafilomycin A1 (BafA1); and were subsequently labeled for acidic vesicles using Lysotracker Red and for viability using Calcein AM. Each experiment was repeated three times with similar results. Scale bar=100 microns.

FIGS. 8A-8F show the cytoprotective effects of Bafilomycin A1 are Bax-independent in CGNs. CGNs were isolated and cultured in vitro for 4 days as described in the Methods section. Control (CTL) and chloroquine (CQ; 20 μM)-treated, wild type (WT) and Bax-deficient (KO) CGNs were assessed for viability (FIGS. 8A and 8C) and fractional caspase-3-like activity (a marker of apoptosis) (FIGS. 8B and 8D) in the presence of 1 nM (FIGS. 8A and 8B) or 10 nM (FIGS. 8C and 8D) Bafilomycin A1 (BafA1). At least three independent experiments of at least three replicates represent each concentration and genotype tested. A representative western blot (FIG. 8E) shows cleaved caspase-3 and processing of LC3 in WT and KO lysates following 24 hours treatment with CTL or chloroquine (CQ; 20 μM). NT=no treatment, 0 hours. A representative western blot (FIG. 8F) shows bafilomycin A1 at 1 nM (BafA1-low) inhibits the increase in p18 Bax levels induced by chloroquine (CQ; 20 μM) in WT CGN cells following 24 hours of treatment with the indicated compounds. For FIGS. 8A and 8B, significant effects of genotype versus treatment were assessed via two-factor ANOVA and Bonferonni's post hoc test and significant treatment effects were assessed via one-factor ANOVA and Bonferonni's post hoc test; p<0.05 (*compared to respective WT treatment pair; ^{†}compared to genotype-matched CTL; ^{‡}compared to genotype-matched treatment with 1 nM Bafilomycin A1; ^{#}compared to genotype-matched treatment with CQ). For FIGS. 8C and 8D, significant effects of genotype versus treatment were assessed via two-factor ANOVA and Bonferonni's post hoc test and significant treatment effects were assessed via unpaired, two-tailed t-test; p<0.05 (*compared to respective WT treatment pair; ^{†}compared to genotype-matched CTL).

FIGS. 9A-9D show that neither caspase-3 deficiency nor pharmacological inhibition of caspases prevents chloroquine-induced death in CGNs. CGNs were isolated and cultured in vitro for 4 days as described in the Methods section. Cell viability (FIGS. 9A and 9C) and fractional caspase-3-like activity (a marker of apoptosis) (FIGS. 9B and 9D) were measured 24 hours following treatment with chloroquine (CQ) or control (CTL) in wild type (WT) and caspase-3-deficient (KO) CGNs (FIGS. 9A and 9B) or in CGNs co-treated with the broad spectrum caspase inhibitor BOC-aspartyl(Ome)-fluoromethyl ketone (BAF, 150 μM; FIGS. 9C and 9D). At least three independent experiments of at least three replicates represent each concentration and genotype tested. For FIGS. 9A and 9B, significant effects of genotype were assessed via two-tailed, unpaired t test; p<0.05 (*compared to CTL). For FIGS. 9C and 9D, significant effects of treatment were assessed via one-factor ANOVA and Bonferonni's post hoc test; p<0.05 (*compared to CTL; ^{†}compared to CQ).

FIG. 10 shows a proposed model indicating the potential concentration-dependent effects of Bafilomycin A1 on neuronal cell death. At low concentrations, Bafilomycin A1 is postulated to inhibit cell death signals resulting from lysosomal dysfunction or autophagic stress.

### DETAILED DESCRIPTION

The present disclosure describes the use of macrolide antibiotics to inhibit neurodegeneration. A number of pathological conditions are characterized by neurodegeneration and include both early onset and age-related diseases and conditions. The molecular basis for the observed neurodegeneration can vary with the particular pathological condition.

Recent evidence implicates the lysosomal pathway and autophagy (such as but not limited to autophagic stress) in the pathology of neurodegeneration. The lysosomal pathway comprises a dynamic system of cellular organelles which function to recycle cellular components to provide a supply of basic cellular ingredients required to maintain the health of the cells. One component of the lysosomal pathway is autophagy. Autophagy is a tightly regulated process which may be induced by nutritional or trophic deprivation or by other conditions of cellular stress. Autophagy is initiated when a region of the cytoplasm is enclosed by a double membraned vacuole, which is termed the autophagosome. The autophagosome matures into a single membrane phagolysosome and ultimately becomes acidified and acquires proteolytic enzymes after fusing with endosomes or lysosomes to complete the degradative process. Lysosomal pathways, including autophagy, are active during normal cellular development to support changes in cell size, remodeling and morphology. Furthermore, such systems may act as surveillance systems to remove damaged cellular components. However, disruptions in the normal lysosomal pathways or autophagy can generate active death signals that may result in cellular death or abnormal cell function.

Disruptions in normal autophagy can lead to autophagic stress, which is defined morphologically by the accumulation of autophagic vacuoles and biochemically by an increase in levels of LC3-II, a protein that associates specifically with the outer membrane of autophagic vacuoles. In addition, the dysfunction of lysosomal degradation pathways (exemplified in the present disclosure by chloroquine treatment of CGNs as an in vitro model) induces a type of autophagic stress which leads to an increase in autophagic vacuoles. Autophagic stress may lead to subsequent cell death via apoptotic pathways (as measured by an increase in protease activity, such as but not limited to caspase-3 activity), as well as leading to cell death via other types of mechanisms that have been shown to occur concomitantly with and independent of apoptosis as a result of autophagic stress.

Several recent reports describe the impairment of the lysosomal system and autophagic stress in a number of neurological diseases and conditions. Reduction in the capacity of the lysosomal system leads to the aberrant deposition of cellular materials, including but not limited to, proteins and glycoconjugate components. Over time, the aberrant deposition of cellular materials leads to developmental abnormalities characteristic of such diseases and conditions and ultimately neurodegeneration. Furthermore, reduction in the capacity of the lysosomal system and an increase in autophagic stress can lead to cell death as discussed above.

These developmental abnormalities are believed to underlie the neurodegeneration and other symptoms observed in a number of early onset and age-related disease pathologies or conditions, including but not limited to, lysosomal storage disorders (LSDs), Tay-Sach's disease, juvenile neuronal ceroid lipofuscinosis, Niemann-Pick disease, Sandoff's disease, Sanfillippo B syndrome, Alzheimer's disease, frontotemporal dementia, Parkinson's disease, Huntington's disease, FTDP-17, and Lewy body dementia.

Certain macrolide antibiotics, such as but not limited to, bafilomycin A1, have been reported to inhibit vacuolar ATPase at concentrations greater than 1 nM. The inhibition of vacuolar ATPase neutralizes acidic vesicles and organelles. The neutralization of acidic vesicles and organelles prevents the fusion of autophagosomes with lysosomes and disrupts the normal function of the lysosomal system. This disruption of the lysosomal system leads to accumulation of intermediates involved in such system, such as but not limited to autophagosomes, and can lead to autophagic stress and subsequent neurodegeneration.

The present disclosure uses a model system to describe a novel neuroprotective effect of macrolide antibiotics that is independent of the effects on vacuolar ATPase and neutralization of acidic vesicles. The dysfunction of lysosomal degradation pathways is exemplified in the present disclosure by chloroquine treatment of CGNs. Chloroquine treatment of CGNs induces dysfunction of the lysosomal pathway and induces a type of autophagic stress which leads to an increase in autophagic vacuoles, which results in a decrease in cell viability, an increase in caspase-3 activity (a marker of apoptotic activity), and an increase in LC3-II processing (a marker of autophagic stress). The present disclosure describes the use of macrolide antibiotics to prevent neurodegeneration and to treat or prevent disease and conditions which involve neurodegeneration. While not being limited to a specific mechanism of action, in one embodiment, the macrolide antibiotics inhibit neurodegeneration caused by lysosomal dysfunction and/or autophagic stress. The present disclosure contemplates the use of any macrolide antibiotic and derivatives thereof. In a specific embodiment, the macrolide antibiotics include bafilomycin A1, bafilomycin B1 and concanamycin, or derivatives thereof. In a further specific embodiment, the macrolide antibiotic is bafilomycin A1, or a derivative thereof.

### DEFINITIONS

The terms “prevention”, “prevent”, “preventing”, “suppression”, “suppress” and “suppressing” as used herein refer to a course of action (such as administering a compound or pharmaceutical composition) initiated prior to the onset of a symptom, aspect, or characteristics of a disease or condition so as to prevent or reduce such symptom, aspect, or characteristics. Such preventing and suppressing need not be absolute to be useful.

The terms “treatment”, “treat” and “treating” as used herein refers a course of action (such as administering a compound or pharmaceutical composition) initiated after the onset of a symptom, aspect, or characteristics of a disease or condition so as to eliminate or reduce such symptom, aspect, or characteristics. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a disease or condition that is treatable by a method or compound of the disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a disease or condition that is preventable by a method or compound of the disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

The term “therapeutically effective amount” as used herein refers to an amount of a compound, either alone or as a part of a pharmaceutical composition, that is capable of having any detectable, positive effect on any symptom, aspect, or characteristics of a disease or condition. Such effect need not be absolute to be beneficial.

The term “macrolide antibiotic” as used herein refers to compounds comprising a lactone ring (referred to as the macrolide ring) to which one or more substituents, such as but not limited to, deoxy sugars, are attached. Macrolide antibiotics include the plecomacrolide antibiotics. Exemplary plecomacrolide antibiotics include, but are not limited to, bafilomycin A1, bafilomycin B1 and concanamycin.

The term “neurodegeneration” as used herein refers to any condition in which neuronal structure or function is impaired and which impairment may be, at least partially reversed or prevented by a macrolide antibiotic as described in the present application.

The term “pharmaceutically acceptable derivative” means any pharmaceutically acceptable salt, ester, salt of an ester, solvate or other derivative of a macrolide antibiotic of the present disclosure that, upon administration to a recipient, is capable of providing (directly or indirectly) a macrolide antibiotic of the disclosure or a metabolite or residue thereof. Particularly favored derivatives are those that increase the bioavailability of the macrolide antibiotics of the disclosure when such macrolide antibiotics are administered to a patient (e.g., by allowing an orally administered compound to be more readily absorbed into the blood), enhance delivery of the macrolide antibiotic to a given biological compartment, increase solubility to allow administration by injection, alter metabolism or alter rate of excretion. In one embodiment, the derivative is a prodrug. Exemplary prodrug forms of macrolide antibiotics are described in U.S. Pat. No. 6,809,080.

The term “pharmaceutically acceptable salt(s)”, unless otherwise indicated, includes salts of acidic or basic groups that may be present in the macrolide antibiotics of the present disclosure.

### Methods of Treatment and Prevention

The present disclosure describes the use of macrolide antibiotics to inhibit and/or prevent neurodegeneration. The present disclosure provides for methods to treat and/or prevent diseases or conditions characterized by neurodegeneration in a subject in need of such treatment and/or prevention. The present disclosure also provides for methods to treat and/or prevent diseases and conditions which depend on a neurodegenerative process in their etiology in a subject in need of such treatment and/or prevention.

In one embodiment, the teachings of the present disclosure provide for the treatment of a disease or condition characterized by neurodegeneration or which depend on a neurodegenerative process in their etiology in a subject in need of such treatment. Such diseases or conditions include, but are not limited to, lysosomal storage disorders (LSDs), Tay-Sach's disease, juvenile neuronal ceroid lipofuscinosis, Niemann-Pick disease, Sandoff's disease, Sanfillippo B syndrome, Alzheimer's disease, frontotemporal dementia, Parkinson's disease, Huntington's disease, FTDP-17, and Lewy body dementia. In a specific embodiment, the disease or condition is characterized by a lysosomal dysfunction and/or autophagic stress.

The method of treatment comprises the steps of identifying a subject in need of such treatment and initiating in said subject a treatment regimen comprising administering at least one macrolide antibiotic, or a pharmaceutically acceptable derivative thereof. The macrolide antibiotic may be a plecomacrolide antibiotic. In a specific embodiment, the macrolide antibiotic is bafilomycin A1, bafilomycin B1, concanamycin or combinations thereof. In a specific embodiment, the macrolide antibiotic is administered in a therapeutically effective amount. Such administration of a macrolide antibiotic would thereby treat the disease or condition. The treatment of neurodegeneration may comprise, at least in part, inhibition of neuronal cell death, inhibition of aberrant neuronal pathology (such as but not limited to, neuritic degeneration), inhibition of a death signal generated by lysosomal dysfunction and/or inhibition of a death signal generated by autophagic stress. As discussed above, the treatment need not be absolute to provide benefit in the treatment methods disclosed. In one embodiment, neurodegeneration is inhibited at least 1%, 5%, 10%, 20%, 30%, 40%, 50% or greater as compared to the neurodegeneration observed without treatment.

In an alternate embodiment, the teachings of the present disclosure provide for the prevention of neurodegeneration and/or a disease or condition characterized by neurodegeneration or which depend on a neurodegenerative process in their etiology in a subject in need of such treatment. Such diseases or conditions include, but are not limited to, lysosomal storage disorders (LSDs), Tay-Sach's disease, juvenile neuronal ceroid lipofuscinosis, Niemann-Pick disease, Sandoff's disease, Sanfillippo B syndrome, Alzheimer's disease, frontotemporal dementia, Parkinson's disease, Huntington's disease, FTDP-17, and Lewy body dementia. In a specific embodiment, the disease or condition is characterized by a lysosomal dysfunction and/or autophagic stress.

The method of prevention comprises the steps of identifying a subject in need of such prevention and initiating in said subject a prevention regimen comprising administering at least one macrolide antibiotic, or a pharmaceutically acceptable derivative thereof. The macrolide antibiotic may be a plecomacrolide antibiotic. In a specific embodiment, the macrolide antibiotic is bafilomycin A1, bafilomycin B1 or concanamycin. In a specific embodiment, the macrolide antibiotic is administered in a therapeutically effective amount. Such administration of a macrolide antibiotic would thereby prevent the disease or condition. The treatment of neurodegeneration may comprise, at least in part, inhibition of neuronal cell death, inhibition of aberrant neuronal pathology (such as but not limited to, neuritic degeneration), inhibition of a death signal generated by lysosomal dysfunction and/or inhibition of a death signal generated by autophagic stress. As discussed above, the prevention need not be absolute to provide benefit in the prevention methods disclosed. In one embodiment, neurodegeneration is inhibited at least 1%, 5%, 10%, 20%, 30%, 40%, 50% or greater as compared to the neurodegeneration observed without prevention.

As described herein, the macrolide antibiotics are shown to reduce neurodegeneration. In one embodiment, the neurodegeneration is caused, at least in part, by lysosomal dysfunction or autophagic stress. For example, the present disclosure shows that certain concentrations of a plecomacrolide antibiotic, bafilomycin A1, inhibits neurodegeneration (assayed by cell death, caspase-3 activity and LC3 processing) caused by chloroquine in CGNs. As discussed above, chloroquine induces dysfunction of the lysosomal pathway and induces a type of autophagic stress which leads to an increase in autophagic vacuoles, and can be used as a model system for lysosomal dysfunction and autophagic stress. Without being bound to alternate mechanisms of action, the macrolide antibiotics may block a death signal generated by lysosomal dysfunction and/or autophagic stress, thereby restoring normal cellular function.

Furthermore, chloroquine and fluoroquinolones (such as but not limited to, moxifloxacin, ciprofloxacin, ofloxacin, levofloxacin, lomefloxacin, norfloxacin, enoxacin, gatifloxacin, and sparfloxacin) have been shown to prevent acidification of vesicles and disrupt mitochondrial membrane potential which results in apoptosis and/or cell death. Drugs such as chloroquine are used to treat diseases and conditions such as malaria, rheumatoid arthritis and autoimmune diseases, but are reported to cause side effects, including but not limited to, neuropathy and impairment of the optic nerve, which may limit their effectiveness. Fluoroquinolones are used to treat bone and joint infections, skin infections, urinary tract infections, inflammation of the prostate, serious ear infections, bronchitis, pneumonia, tuberculosis, some sexually transmitted diseases (STDs), and some infections that affect people with AIDS. By preventing the impact of such drugs on the lysosomal and autophagic systems (such as by inhibition of neuronal cell death, inhibition of aberrant neuronal pathology, inhibition of a death signal generated by lysosomal dysfunction and/or inhibition of a death signal generated by autophagic stress), the side effects of such drugs may be reduced without reducing the beneficial effects.

Therefore, the present disclosure also provides methods of treatment to alleviate or prevent the detrimental effects of drugs which act, at least in part, on the lysosomal or autophagy pathways by administering a therapeutically effective amount of at least one macrolide antibiotic, or a pharmaceutically acceptable derivative thereof, in combination with such drug. The macrolide antibiotics of the present disclosure may be used with any drug which provides beneficial effects but also negatively impacts the lysosomal or autophagy pathways. The treatment may comprise, at least in part, inhibition of neuronal cell death, inhibition of aberrant neuronal pathology (such as but not limited to, neuritic degeneration), inhibition of a death signal generated by lysosomal dysfunction and/or inhibition of a death signal generated by autophagic stress.

The methods of the treating and/or preventing discussed herein may also comprise further administering of one or more additional therapeutic agents in combination with the macrolide antibiotics described above.

### Methods of Identification

Furthermore, the teachings of the present disclosure can be used to identify compounds that treat and/or prevent a disease or condition characterized by neurodegeneration or which depend on a neurodegenerative process in their etiology. The compounds identified may thus be useful in the treatment and/or prevention methods described above. Such compounds may be small-molecule pharmaceuticals, peptides, biologics, various non-coding RNAs, antisense molecules and antibodies.

The methods or assays for identifying such compounds comprise providing a cell line or model system in which the lysosomal or autophagy system is at least partially impaired or which can be induced to become impaired, incubating said cells or model with a candidate compound, determining a characteristic of said model system in the presence of said compound and in the absence of said compound and determining whether said characteristic is increased or decreased by the presence of said compound. Such a characteristic may be any characteristic that is measurable using analytical techniques currently known in the art. Exemplary characteristic include, but are not limited to, protease activity (such as but not limited to caspase activity, particularly caspase-3 activity), biochemical markers of apoptosis (such as but not limited to caspase-3) activity or autophagy (such as but not limited to LC3 processing), cell viability, acidification of vesicles or other markers or test described herein or known in the art. The compound may increase or decrease said characteristic as describe herein.

### Pharmaceutical Compositions

The macrolide antibiotics described above for use in the methods described herein may be administered alone or as a pharmaceutical composition formulated by any method known in the art. Certain exemplary methods for preparing the compounds and pharmaceutical compositions are described herein and should not be considered as limiting examples. Furthermore, the compounds or pharmaceutical compositions may be administered to the subject as is known in the art and determined by a healthcare provider. Certain modes of administration are provided herein and should not be considered as limiting examples. Furthermore, the compound or pharmaceutical composition may be administered with other agents in the methods described herein. Such other agents may be agents that increase the activity of the compounds disclosed, such as by limiting the degradation or inactivation of the compounds disclosed or increasing the absorption or activity of the compounds disclosed.

The compounds and pharmaceutical compositions described can be used in the form of a medicinal preparation, for example, in aerosol, solid, semi-solid or liquid form which contains the compounds disclosed as an active ingredient. In addition, the pharmaceutical compositions may be used in an admixture with an appropriate pharmaceutically acceptable carrier. Such pharmaceutically acceptable carriers include, but are not limited to, organic or inorganic carriers, excipients or diluents suitable for pharmaceutical applications. The active ingredient may be compounded, for example, with the usual non-toxic pharmaceutically acceptable carriers, excipients or diluents for tablets, pellets, capsules, inhalants, suppositories, solutions, emulsions, suspensions, aerosols and any other form suitable for use. Pharmaceutically acceptable carriers for use in pharmaceutical compositions are well known in the pharmaceutical field, and are described, for example, in Remington: The Science and Practice of Pharmacy Pharmaceutical Sciences, Lippincott Williams and Wilkins (A. R. Gennaro editor, 20^{th} edition). Such materials are nontoxic to the recipients at the dosages and concentrations employed and include, but are not limited to, water, talc, gum acacia, gelatin, magnesium trisilicate, keratin, colloidal silica, urea, buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, lactose, mannitol, glucose, mannose, dextrins, potato or corn starch or starch paste, chelating agents such as EDTA, sugar alcohols such as mannitol or sorbitol, counterions such as sodium and/or nonionic surfactants such as Tween, Pluronics or polyethyleneglycol. In addition, the pharmaceutical compositions may comprise auxiliary agents, such as, but not limited to, taste-enhancing agents, stabilizing agents, thickening agents, coloring agents and perfumes.

Pharmaceutical compositions may be prepared for storage or administration by mixing a compound of the present disclosure having a desired degree of purity with physiologically acceptable carriers, excipients, stabilizers, auxiliary agents etc. as is known in the pharmaceutical field. Such pharmaceutical compositions may be provided in sustained release or timed release formulations.

The pharmaceutical compositions may be administered orally in solid dosage forms, such as capsules, tablets, and powders, or in liquid dosage forms, such as elixirs, syrups and suspensions. It can also be administered parenterally, in sterile liquid dosage forms. Furthermore, pharmaceutical compositions may be administered parenterally by transmucosal delivery via solid, liquid or aerosol forms of transdermally via a patch mechanism or ointment. Various types of transmucosal administration include respiratory tract mucosal administration, nasal mucosal administration, oral transmucosal (such as sublingual and buccal) administration and rectal transmucosal administration.

For preparing solid compositions such as, but not limited to, tablets or capsules, the pharmaceutical compositions may be mixed with an appropriate pharmaceutically acceptable carriers, such as conventional tableting ingredients (lactose, sucrose, mannitol, corn starch, potato starch, alginic acid, microcrystalline cellulose, acacia, gelatin, gums, colloidal silicon dioxide, croscarmellose sodium, talc, sorbitol, stearic acid magnesium stearate, calcium stearate, zinc stearate, stearic acid, dicalcium phosphate other excipients, colorants, diluents, buffering agents, disintegrating agents, moistening agents, preservatives, flavoring agents, and pharmacologically compatible carriers) and diluents (including, but not limited to, water, saline or buffering solutions) to form a substantially homogenous composition. The substantially homogenous composition means the components (a compound as described herein and a pharmaceutically acceptable carrier) are dispersed evenly throughout the composition so that the composition may be readily subdivided into equally effective unit dosage forms such as tablets, pills and capsules. The solid compositions described may be coated or otherwise compounded to provide a dosage form affording the advantage of prolonged action. For example, the tablet or pill can comprise an inner dosage an outer dosage component, the latter being in the form of an envelope over the former. The two components can be separated by an enteric layer which serves to resist disintegration in the stomach and permits the inner component to pass intact through the stomach or to be delayed in release. A variety of materials can be used for such enteric layers or coatings such materials including a number of polymeric acids and mixtures of polymeric acids with such materials as shellac, cetyl alcohol and cellulose acetate. The active compounds may also be formulated in rectal compositions such as suppositories or retention enemas, e.g., containing conventional suppository bases such as cocoa butter or other glycerides. The solid compositions may also comprise a capsule, such as hard- or soft-shelled gelatin type containing, for example, surfactants, lubricants, and inert fillers, such as lactose, sucrose, calcium phosphate, and corn starch.

For intranasal administration, intrapulmonary administration or administration by other modes of inhalation, the pharmaceutical compositions may be delivered in the form of a solution or suspension from a pump spray container or as an aerosol spray presentation from a pressurized container or nebulizer, with the use of a suitable propellant (e.g., dichlorodifluoromethane, trichlorofluoromethane, dichlorotetrafluorocthane, nitrogen, propane, carbon dioxide or other suitable gas) or as a dry powder. In the case of an aerosol or dry powder format, the amount (dose) of the compound delivered may be determined by providing a valve to deliver a metered amount.

Liquid forms may be administered orally, parenterally or via transmucosal administration. Suitable forms for liquid administration include aqueous solutions, suitably flavored syrups, aqueous or oil suspensions, and flavored emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenizoates or sorbic acid); and artificial or natural colors and/or sweeteners. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. For buccal or sublingual administration, the composition may take the form of tablets or lozenges formulated in conventional manners. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds disclosed (whether alone or in pharmaceutical compositions) may be formulated for parenteral administration. Parenteral administration includes, but is not limited to, intravenous administration, subcutaneous administration, intramuscular administration, intradermal administration, intrathecal administration, intraarticular administration, intracardiac administration, retrobulbar administration and administration via implants, such as sustained release implants.

The pharmaceutical compositions may be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutically acceptable carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4th ed., 622-630 (1986).

The pharmaceutical compositions are administered in therapeutically effective amount. The therapeutically effective amount will, of course, vary depending upon known factors, such as the pharmacodynamic characteristics of the particular compound and its mode and route of administration; the age, health and weight of the subject; the severity and stage of the disease state or condition; the kind of concurrent treatment; the frequency of treatment; and the effect desired. The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or diseases, in particular chronic conditions or diseases, may require prolonged treatment involving multiple administrations.

### Results

In the following results, the methods used were those methods specified in the Methods section of the present disclosure and the references cited therein.

### Chloroquine Induces Neuronal Cell Death

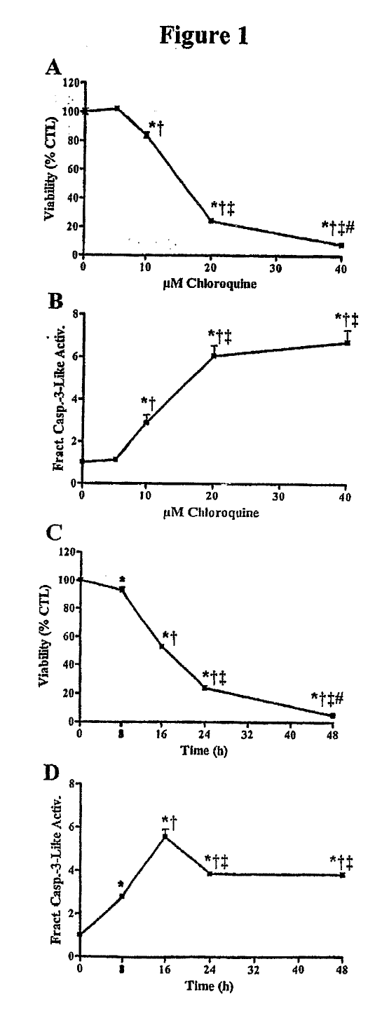

In order to determine the effect of chloroquine on neuronal cells and neurodegeneration, cerebellar granule neurons (CGNs) were cultured for 4 days and treated with 5-40 μM chloroquine for 24 hours in vitro and their viability and fractional caspase-3-like activity (an enzyme activity associated with apoptotic cell death) were measured 16-24 hours later (FIGS. 1A and 1B). Chloroquine produced a concentration-dependent decrease in viability (FIG. 1A) and an increase in caspase-3-like activity (FIG. 1B), with significant effects observed at concentrations of chloroquine ≧10 μM. Chloroquine decreased viability to 24% at 20 μM and to 8% at 40 μM (FIG. 1A) while a 6-7 fold increase in caspase-3-like activity was maximal at 20-40 μM (FIG. 1B). For all subsequent experiments described herein, a concentration of 20 μM chloroquine was used. Treatment with cycloheximide (0.1-1 mg/ml), an inhibitor of protein synthesis, did not alter the magnitude of cell death induced by chloroquine (data not shown), which suggests that the deleterious effects of chloroquine were not dependent on de novo protein synthesis.

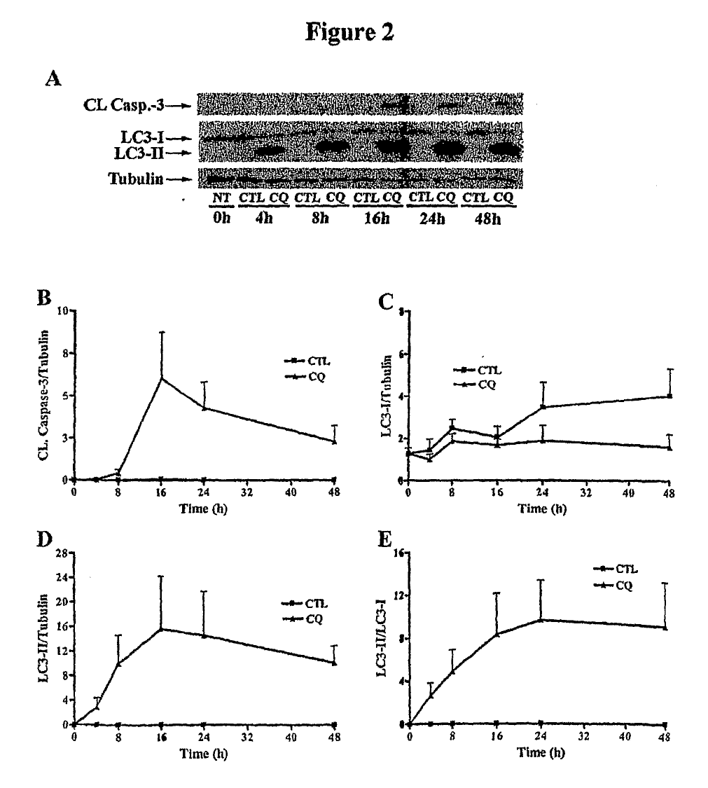

Time course studies were performed to determine the temporal induction of cell death and caspase-3-like activity by chloroquine (FIGS. 1C and 1D) in CGNs. In these experiments, CGNs were cultured for 4 days in vitro and measurements were obtained up to 48 hours after chloroquine treatment at 20 μM. Viability was slightly lower after 8 h (93%) and continued to decrease significantly over the next 40 hours (FIG. 1C) at a concentration of 20 μM chloroquine. Caspase-3-like activity increased significantly 8 hours following treatment with chloroquine and was maximal at 16 hours (6-fold induction), and remained at high levels from 24-48 hours (FIG. 1D) at a concentration of 20 μM chloroquine. The temporal pattern of cleaved or “active” caspase-3 (CL caspase-3) in CGNs was confirmed by western blot analysis (FIGS. 2A and 2B), which was evident at low levels by 8 hours and was most robust at 16 hours following treatment with 20 μM chloroquine. In FIGS. 2A-2E, CQ indicates chloroquine treatment, CTL indicates control and NT indicates no treatment.

To determine the effects of chloroquine on autophagosomes, the processing of LC3 was assessed via western blot (FIGS. 2A and 2C-E). CGNs were cultured for 4 days in vitro and measurements were obtained up to 48 hours after chloroquine treatment at 20 μM. LC3 is a microtubule-associated protein that upon processing from its cytoplasmic form (LC3-I) inserts via covalent lipidation into the inner and outer limiting membranes of autophagosomes as LC3-II^{9, 10}. Levels of the 18 kDa LC3-I appeared lower at 24-48 hours following treatment with chloroquine in comparison to levels measured at those times in control samples (FIGS. 2A and 2C). LC3-II, the 16 kDa form of LC3 specific for membranes of autophagosomes¹⁰, was elevated within 4 hours after exposure with chloroquine and remained high through 48 hours (FIGS. 2A and 2D), an effect that was virtually absent in control samples. This increase in LC3-II was also reflected by an increase in the ratio of autophagosome-bound LC3-II to that of cytosolic LC3-I (FIG. 2E). The increase in autophagosome-bound LC3-II suggests that chloroquine interrupts the lysosomal pathway by increasing the number of autophagosomes in the CGNs and disrupts normal autophagy (i.e., inducing autophagic stress).

### Bafilomycin A1 Attenuates Chloroquine-induced Apoptosis

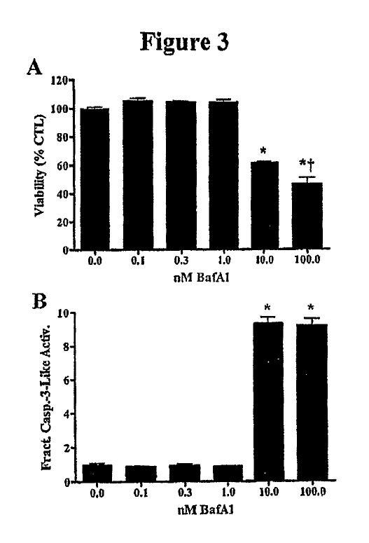

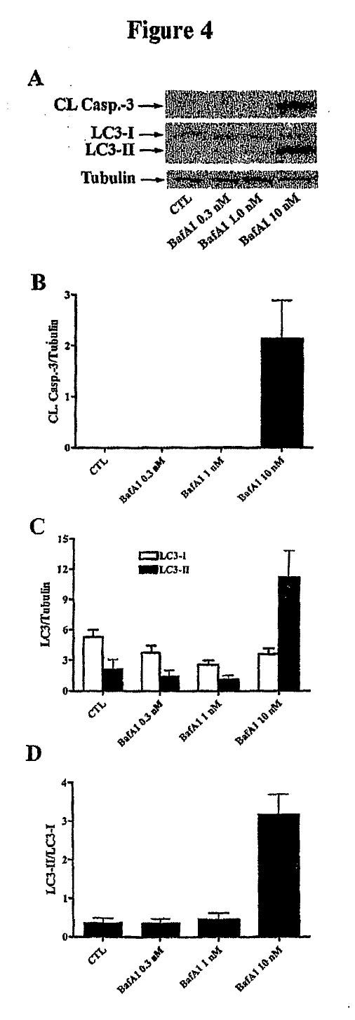

The effects of bafilomycin A1 on CGNs were assessed after 24 hours exposure to bafilomycin A1 (FIGS. 3A and 3B). In these experiments, CGNs were cultured for 4 days in vitro and measurements were obtained 24 hours after addition of bafilomycin A1. By itself, bafilomycin A1 did not decrease viability or induce caspase-3-like activity at concentrations ≦1 nM, but significantly decreased viability and increased caspase-3-like activity at concentrations ≧10 nM (FIGS. 3A and 3B). Western blot analysis was performed to confirm the effects of bafilomycin A1 on the activity of caspase-3 and to determine its effects on the processing of LC3 (FIG. 4). An increase in cleaved caspase-3 (CL caspase-3) was only apparent upon treatment with 10 nM bafilomycin A1 (FIGS. 3B, 4A and 4B). Treatment with bafilomycin A1 at any concentration did not change levels of LC3-I, but 10 nM bafilomycin A1 induced a dramatic increase in levels of LC3-II (FIGS. 4A and 4C), which was also reflected by an increase in the ratio of LC3-II/LC3-I (FIG. 4D).

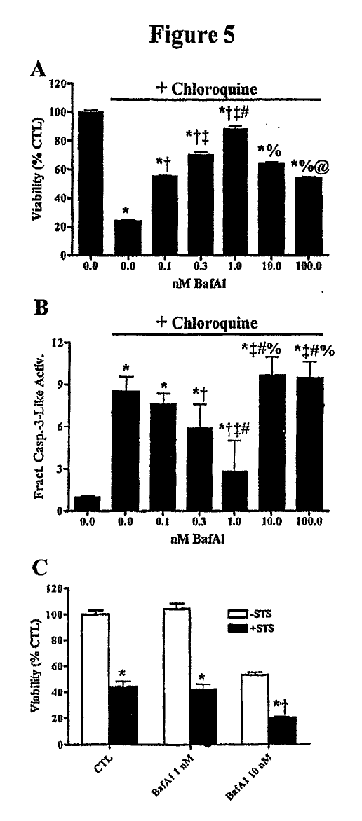

The effects of bafilomycin A1 were also assessed in the presence of a toxic dose of chloroquine (20 μM) (FIG. 5). In these experiments, CGNs were cultured for 4 days in vitro and measurements were obtained 24 hours after addition of bafilomycin A1 and chloroquine at the concentrations indicated. Bafilomycin A1 significantly attenuated the chloroquine-induced decrease in viability at all concentrations tested, and attenuation was maximal at 1 nM (88% with bafilomycin A1 versus 24% without; FIG. 5A). Although 10-100 nM bafilomycin A1 decreased viability alone (FIG. 3A), these concentrations still attenuated the larger decrease in viability induced by chloroquine (FIG. 5A). The chloroquine-induced increase in caspase-3-like activity was reduced only upon the co-addition of 0.3-1 nM bafilomycin A1, and was maximal at 1 nM (3-fold reduction; FIG. 5B). Bafilomycin B1 and chloroquine also attenuated the chloroquine-induced (20 μM) decrease in viability at all 1 nM under the conditions above (data not shown).

To determine whether bafilomycin A1 affected the toxicity of a classical apoptosis-inducing stimulus, CGNs were treated for 24 h with 0.1 μM staurosporine (STS) (FIG. 5C), a concentration that significantly increases caspase-3-like activity in the CGN culture system (data not shown). Treatment with staurosporine for 24 hours reduced viability to an average of 44%, an effect that was not altered by co-administration with 1 nM bafilomycin A1. Treatment with 10 nM bafilomycin A1, however, further decreased the viability of staurosporine-treated CGNs to 20%.

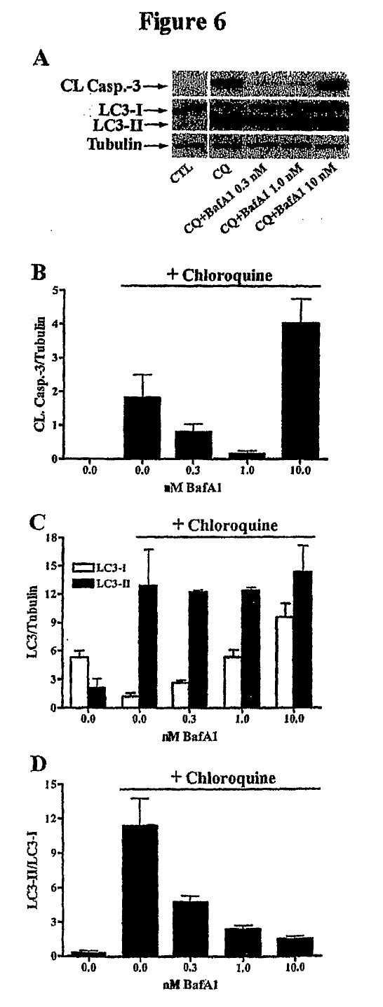

Western blot analysis was performed to confirm the effects of bafilomycin A1 on the activity of caspase-3 and to determine its effects on the processing of LC3, in the presence of chloroquine (20 μM) (FIGS. 6A-D). In these experiments, CGNs were cultured for 4 days in vitro and measurements were obtained 24 hours after addition of bafilomycin A1 and chloroquine at the concentrations indicated. The chloroquine-induced increase in cleaved caspase-3 (CL caspase-3) was attenuated by co-treatment with ≦1 nM bafilomycin A1 (FIGS. 6A and 6B). Bafilomycin A1 did not attenuate chloroquine-induced levels of LC3-II but appeared to increase levels of LC3-I (FIG. 6C), which is reflected by a reduction in the ratio of LC3-II to LC3-I at all concentrations of bafilomycin A1 tested (FIGS. 6A and 6D).

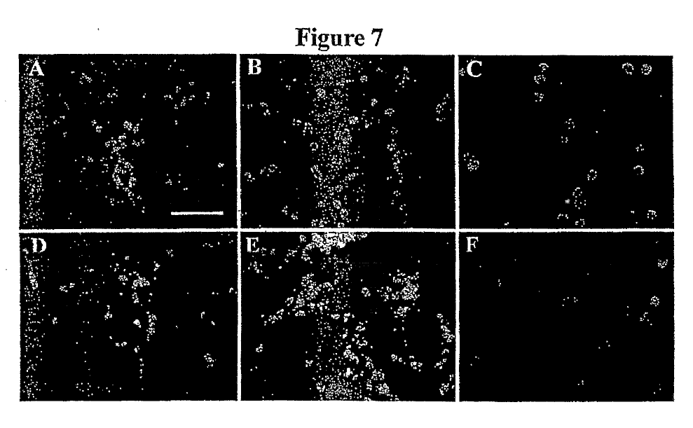

Vacuolar acidification was measured following 24 h treatment with chloroquine (20 μM) and/or bafilomycin A1 by incubation with Lysotracker Red (LTR), in the presence of the viability marker calcein AM (FIG. 7). In these experiments, CGNs were cultured for 4 days in vitro and measurements were obtained 24 hours after addition of bafilomycin A1 and chloroquine at the concentrations indicated. By itself, 1 nM bafilomycin A1 did not alter the staining pattern of LTR nor did it reduce viability in comparison to control cells (FIGS. 7A and 7B). In contrast, treatment with 10 nM bafilomycin A1 not only prevented any detection of vacuolar acidification by LTR but also decreased numbers of viable CGNs (FIG. 7C), an effect that was similar in the presence or absence of chloroquine (FIGS. 7C and 7F). Treatment with chloroquine decreased numbers of viable CGNs (FIG. 7D). However, this reduced population of CGNs that survived 24 hours treatment with chloroquine exhibited a greater intensity of LTR staining than that observed in control cells (FIG. 7D). The co-addition of 1 nM bafilomycin A1 and chloroquine increased numbers of viable neurons that also exhibited a concomitant increase in LTR staining intensity, in comparison to treatment with chloroquine or bafilomycin A1 alone (FIG. 7E).

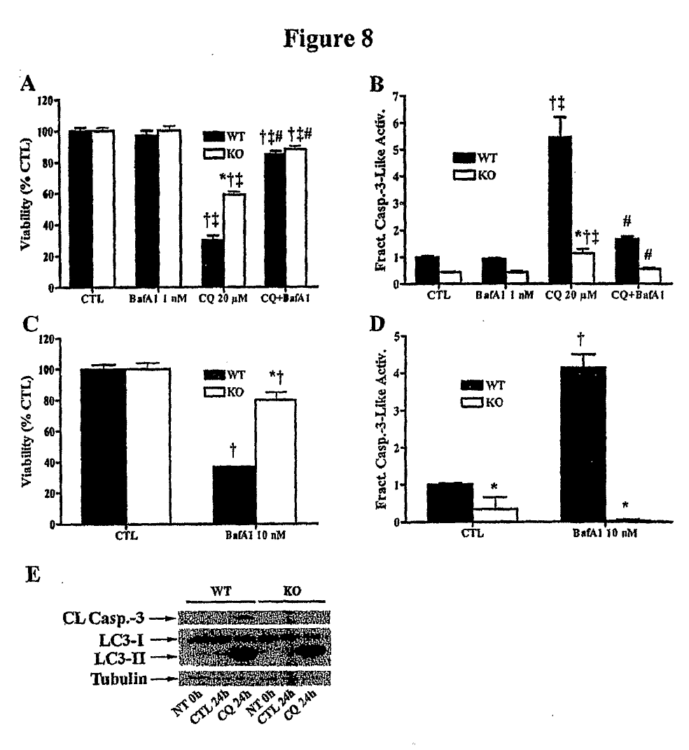

To assess the role of Bcl-2 family members in this process, cultures were prepared from Bax-deficient CGNs (designated KO) (FIGS. 8A-E). In these experiments, CGNs (wild-type, designated WT, and Bax deficient, designated KO) were cultured for 4 days in vitro and measurements were obtained 24 hours after addition of bafilomycin A1 and chloroquine at the concentrations indicated. Bax is a member of the pro-apoptotic Bcl-2 family and has been postulated to play a role in chloroquine-induced death and to be involved in cell death resulting from lysosomal dysfunction and autophagic stress. The targeted deletion of Bax attenuated the chloroquine-induced (20 μM) decrease in viability (FIG. 8A) and increase in caspase-3-like activity (FIG. 8B), measured at 24 hours as compared to wild-type (WT), but did not provide any greater protection than that achieved with 1 nM bafilomycin A1 (FIGS. 8A and 8B). Conversely, Bax deficiency prevented the reduction in viability and increase in caspase-3-like activity that was induced by treatment for 24 hours with 10 nM bafilomycin A1 (FIGS. 8C and 8D). The effects of targeted deletion of Bax on the activity of caspase-3 were confirmed via western blot (FIG. 8E). Furthermore, western blot analysis indicated that Bax deficiency did not prevent the chloroquine-induced increase in LC3-II (FIG. 8E).

Bax can be cleaved from a 21 kDa precursor (p21) to an 18 kDa fragment (p18) by the action of calpain⁴¹. The p18 form of Bax has been shown to enhance its cell death function at the level of the mitochondria. FIG. 8F shows that in CGNs chloroquine (20 μM for 24 hours) and bafilomycin A1 (10 mM for 24 hours) increased the amount of p18 Bax. This increase in p18 Bax levels was dramatically attenuated by 1 nM bafilomycin A1. This suggests that the macrolide antibiotics may induce their neuroprotective effects, at least in part, via attenuation of death signals (such as but not limited to the induction of calpain activity) that activate p18 Bax via a mechanism apart from apoptosis. The cleavage of Bid (another Bcl-2 family member) was not increased by chloroquine, suggesting that Bid activation is not involved in the chloroquine induced effects in CGNs (data not shown).

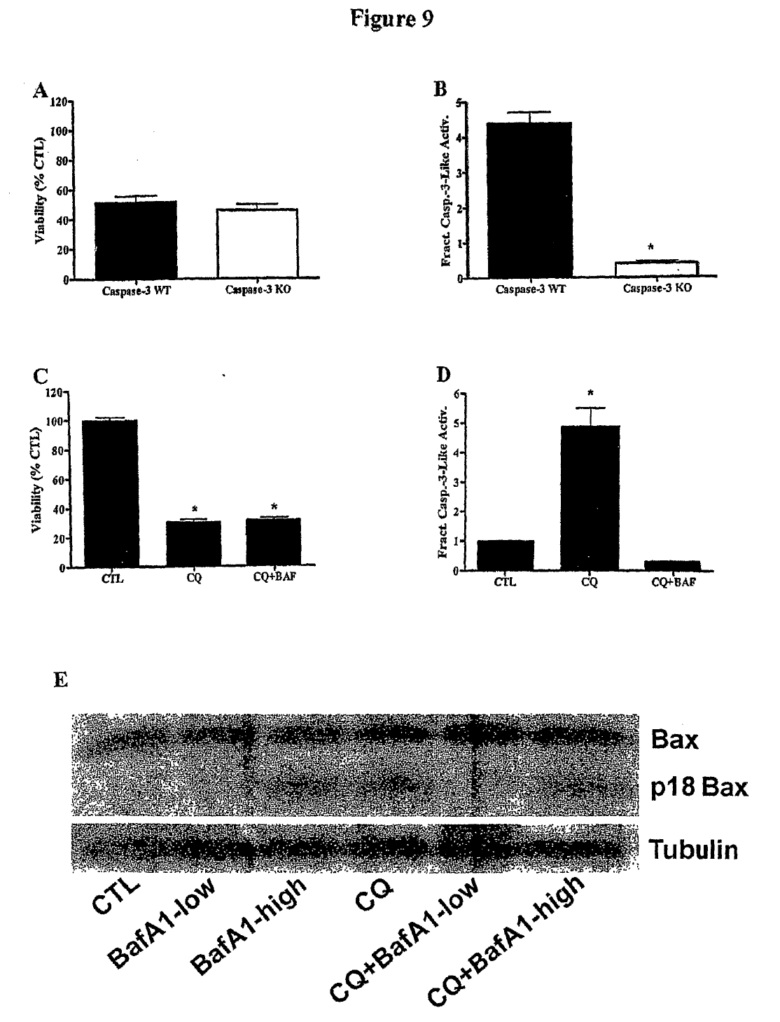

Since chloroquine induced a dramatic increase in the activity of caspase-3, cultures of CGNs were prepared from caspase-3-deficient mice to determine the relative positioning of this effector caspase in the pathway of cell death induced by chloroquine (20 μM for 24 hours). The absence of caspase-3 did not prevent the chloroquine-induced decrease in viability (FIG. 9A), although a significant attenuation in caspase-3-like activity was observed (FIG. 9B). To determine if other caspases, e.g. caspase-9, may be critical for chloroquine-induced death, wild-type CGNs were treated with BOC-aspartyl(Ome)-fluoromethyl ketone (BAF), a broad-spectrum caspase inhibitor. Treatment with this inhibitor did not attenuate chloroquine-induced death (20 μM for 24 hours) (FIG. 9C), but did significantly attenuate the chloroquine-induced increase in caspase-3-like activity (FIG. 9D).

The above results show that administration of macrolide antibiotics, such as, but not limited to, bafilomycin A1, bafilomycin B1 and concanamycin, dramatically and significantly attenuated chloroquine-induced cell death in neuronal cells. This effect was characterized by an inverted, U-shaped concentration-dependence. For bafilomycin A1, this effect was maximal at a concentration of 1 nM. Furthermore, 1 nM bafilomycin B1 and concanamycin also inhibited chloroquine induced cell death in neuronal cells. This concentration of bafilomycin A1 did not alter vacuolar acidification or induce the formation of autophagosomes. Furthermore, these concentrations of macrolide antibiotics were shown previously to have minimal effect on the inhibition of vacuolar ATPase in a cell-free system^{21, 22}. However, 10-100 nM bafilomycin A1, despite its ability to decrease in CGN viability and induction of the cell death pathways, inhibited chloroquine-induced death, although not as robustly as with lower concentrations. This is likely due to Bafilomycin A1-dependent induction of the intrinsic apoptotic pathway at ≧10 mM. It is possible that high concentrations of Bafilomycin A1 prevent a distinct, caspase-independent pathway of chloroquine-induced cell death, since chloroquine-induced caspase-3-like activity was maximal in the presence or absence of ≧10 nM bafilomycin A1. Previously, effects of bafilomycin A1 in various cell culture models have typically been reported using concentrations ≧10 nM^{19, 20, 27, 28}.

One nM bafilomycin A1 not only increased the population of CGNs that survived the chloroquine insult but also increased numbers of intensely-stained, LTR-positive acidic vesicles. A transient, robust increase in LTR staining has been described previously upon treatment with chloroquine^{19, 20} that occurs concomitantly with increased numbers of autophagosomes, yet dissipates only in cells exhibiting a subsequent disruption in mitochondrial membrane potential and resultant apoptosis²⁰. In the present study, CGNs exhibiting intensely-staining LTR may represent the population of cells that have survived the 24 hour chloroquine insult and have intact mitochondrial function.

LTR-positive vesicles may represent a population of late autophagosomes that becomes increasingly acidic during their maturation^{32, 33}. By disrupting the function of lysosomes, chloroquine may prevent their fusion with late autophagosomes, resulting ultimately in the accumulation of late autophagosomes. On the other hand, treatment with 10 nM bafilomycin A1 completely prevents LTR staining, both in the presence or absence of chloroquine. Bafilomycin A1, at concentrations that completely prevent vacuolar ATPase, may prevent the fusion and thus maturation of early autophagosomes, thus generating an accumulation of autophagosomes with a higher pH that would make them undetectable with LTR. Biochemically, LC3-II has been documented in both early and late autophagosomes³⁴, which would indicate why both chloroquine and 10 nM BafA1 increased levels of LC3-II yet produced differential staining patterns with LTR.

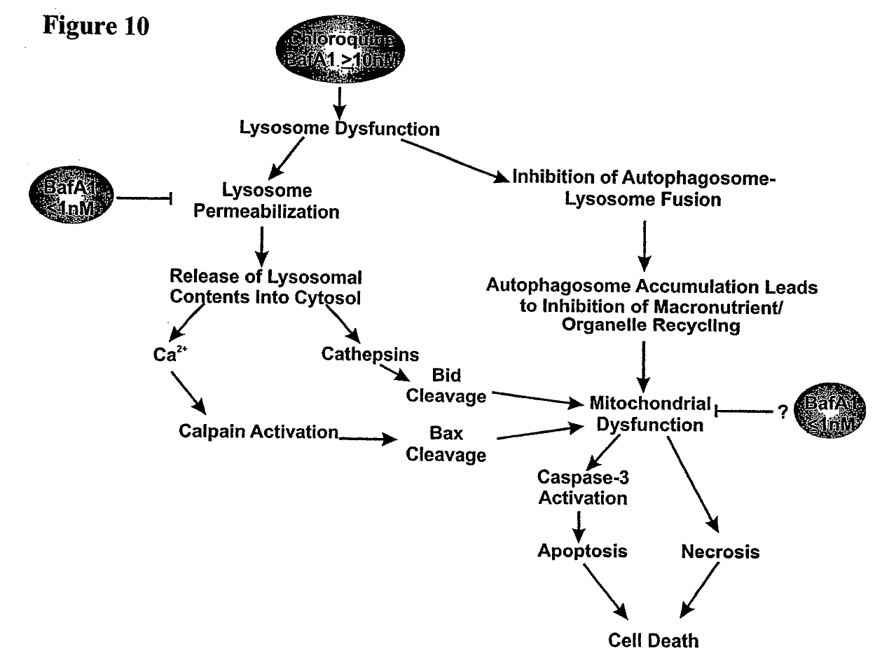

The observed concentration-dependent dichotomy of bafilomycin A1 is highlighted by its effects in the absence of Bax. Although the results clearly indicate that chloroquine-induced death is in part Bax-dependent, the degree of protection afforded by 1 nM bafilomycin A1 against chloroquine was substantially greater than that provided by Bax deficiency. Considering that bafilomycin A1 significantly attenuates activation of caspase-3 and inhibits the formation of p18 Bax, the effects of bafilomycin A1 may lie upstream of Bax and may prevent activation of the intrinsic apoptotic pathway and/or other pathways not involving classical apoptosis but also leading to cellular death (FIG. 10). Since Bax deficiency virtually prevented the chloroquine-induced activation of caspase-3, this additional protective effect of bafilomycin A1 cannot be attributed to its functional inhibition of molecules such as Bak, which shares redundant regulatory functions with Bax^{4, 36}. In contrast, Bax deficiency significantly attenuated the induction of caspase-3-like activity and the reduction in viability resulting from 10 nM bafilomycin A1, which suggests that similar to the effects of chloroquine, the inhibition of autophagy by high concentrations of bafilomycin A1 induces Bax-dependent apoptosis.

Although chloroquine induced a robust activation of caspase-3, the targeted deletion of caspase-3 and the broad pharmacological inhibition of caspases did not prevent chloroquine-induced death. Similar results were obtained previously with cultured telencephalic neurons¹⁸, which suggests that the commitment point for chloroquine-induced neuron death lies upstream of caspase activation. These findings are in contrast to the chloroquine-induced death of HeLa cells, which was attenuated upon treatment with a general caspase inhibitor¹⁹.

The present disclosure reveals a novel, concentration-dependent dichotomy in Bafilomycin A1 function. At macrolide antibiotic concentrations ≦1 nM, the macrolide antibiotics prevent chloroquine induced decrease in cell viability. While not being limited to other alternate mechanistic explanations, the macrolide antibiotics (as illustrated by Bafilomycin A1) may inhibit death signals resulting from lysosomal dysfunction and/or autophagic stress. The data suggests that such inhibition lies upstream of Bax and caspase-3 activation. By blocking such death signals, normal neuronal cellular function is restored and cell death is prevented or attenuated. These results suggest a role for macrolide antibiotics, at low concentrations, as neuroprotective agents. Therefore, by relieving neurodegeneration induced by autophagic stress and/or lysosomal dysfunction, the macrolide antibiotics maintain neuronal cell viability and preserve neuronal cell function.

The foregoing description illustrates and describes the compounds and methods of the present disclosure. Additionally, the disclosure shows and describes only certain embodiments of the compounds and methods but, as mentioned above, it is to be understood that the teachings of the present disclosure are capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. All references cited herein are incorporated by reference as if fully set forth in this disclosure.

### METHODS

### Reagents

Unless otherwise noted, reagents were acquired from Sigma (St. Louis, Mo.).

### Animals

C57BL/6J mice were used in all experiments. The generation of mice deficient in Bax and caspase-3 has been described previously³⁷⁻³⁹. Genetic status was determined by polymerase chain reaction (PCR) analysis of tail DNA extracts as described previously^{38,39}. Mice were cared for in accordance with the guidelines of the NIH Guide for the Care and Use of Laboratory Animals. All animal protocols were approved by the Institutional Animal Care and Use Committee of the University of Alabama at Birmingham.

### Cell Culture

Cerebellar granule neurons (CGNs) were isolated from postnatal day 7 mice as previously described⁴⁰. CGNs were plated in poly-L-lysine coated plates at a density of 1250 cells per mm². 48 well plates were used for viability and caspase activity assays; 100 mm dishes were used for western blot analysis; and 4 well Paradox chamber slides (Agene) were used for analysis of vacuolar acidification.

CGNs were treated in vitro on day 4. The conditioned media was replaced with fresh media with or without chloroquine (5-40 μM final), in the presence or absence of BafA1 (0.1-100 nM final), BOC-aspartyl(Ome)-fluoromethyl ketone (150 μM final; MP Biomedical, Aurora, Ohio) or cycloheximide (0.01-1 μg/ml final). Separate cultures of CGNs were also treated for 24 h with staurosporine (0.1 μM), in the presence or absence of BafA1 (1-10 nM final).

### Measurement of Viability and Caspase-3-Like Activity

Cell viability (via Calcein AM fluorogenic conversion assay; Molecular Probes, Eugene, Oreg.) and caspase-3-like activity (via fluorogenic DEVD cleavage assay) were performed as previously described 40 and were expressed relative to untreated controls.

Labeling of CGNs with LTR

Conditioned media was removed from CGNs grown in 4 well, permanox chamber slides. LTR (0.05 μM; Molecular Probes, Eugene, Oreg.) and Calcein AM (2.5 μg/ml; Molecular Probes, Eugene, Oreg.) were prepared in Locke's Buffer and added to each well for 30 min, 37° C. After this incubation the cells were washed with Locke's Buffer and cover-slipped. Images were captured using a Carl Zeiss Axioskop microscope equipped with epifluorescence.

### Western Blot

Cells were detached from their substrate by incubation for 5 min at 37° C. with Accutase (Innovative Cell Technologies, San Diego, Calif.). Conditioned media and cells were centrifuged (700×g, 5 min, 4° C.). The supernatant was aspirated and re-suspended with 1 ml ice-cold PBS, then centrifuged again (700×g, 5 min, 4° C.). The resultant pellet was re-suspended in lysis buffer containing 25 mM HEPES, 5 mM EDTA, 5 mM MgCl₂, 1% SDS, 1% Triton X-100, 1 mM PMSF, 1% protease inhibitor cocktail and 1% phosphatase inhibitor cocktail (Sigma). Cell lysates were sonicated to shear DNA and then centrifuged (10,000 rpm, 10 min, 4° C.), and the resultant supernatant (cleared whole cell lysates) was transferred to a fresh tube. Protein levels were determined subsequently via BCA assay (Pierce). Equal amounts of protein were resolved via SDS-PAGE and transferred to PVDF. After transfer blots were cut in half at approximately 37 kDa. The lower molecular weight blots were used initially for the detection of active caspase-3 with an antibody raised against the cleaved or active fragment (Cell Signaling), and the upper molecular weight blots were used for detection of β-III tubulin (Santa Cruz) to normalize for protein loading. Blots were blocked for 1 h, RT (5% milk), followed by overnight incubation with primary antibody. Blots were washed with 1×TBS containing 0.1% Tween 20, then incubated with secondary antibody (goat anti-rabbit IgG, Biorad) for 1 h, RT and subsequently washed. Signal was detected using Supersignal chemiluminescence (Pierce). After detection of cleaved caspase-3, the bottom blot was stripped using Restore Western Blot stripping buffer (Pierce) then probed with rabbit anti-LC3 (provided by Uchiyama laboratory), using the same western blot protocol as described above. Blots were scanned for densitometric analysis using Biorad Quantity One® software.

### Statistics

Significant effects of treatment were analyzed either by one-factor ANOVA (if three or more groups) or by unpaired, two-tailed t-test (if two groups). Genotype-specific effects of treatment were analyzed for significance via two-factor ANOVA. Post hoc analysis was conducted using Bonferonni's test. A level of p<0.05 was considered significant.

### REFERENCES

1. Schweichel J U, Merker H J (1973) The morphology of various types of cell death in prenatal tissues. Teratology. 7: 253-266
2. Clarke P G (1990) Developmental cell death: morphological diversity and multiple mechanisms. Anat Embryol (Berl). 181: 195-213
3. Bredesen D E (2000) Apoptosis: overview and signal transduction pathways. J. Neurotrauma. 17: 801-810
4. Putcha G V, Harris C A, Moulder K L, Easton R M, Thompson C B and Johnson E M, Jr. (2002) Intrinsic and extrinsic pathway signaling during neuronal apoptosis: lessons from the analysis of mutant mice. J Cell Biol. 157: 441-453
5. Shacka J J, Roth K A (2005) Regulation of neuronal cell death and neurodegeneration by members of the Bcl-2 family: therapeutic implications. Curr Drug Targets CNS Neurol Disord. 4: 25-39
6. Klionsky D J, Emr S D (2000) Autophagy as a regulated pathway of cellular degradation. Science. 290: 1717-1721
7. Eskelinen E-L (2005) Maturation of Autophagic Vacuoles in Mammalian Cells. Autophagy. 1: 1-10
8. Lum J J, DeBerardinis RJ and Thompson C B (2005) Autophagy in metazoans: cell survival in the land of plenty. Nat Rev Mol Cell Biol. 6: 439-448
9. Ichimura Y, Kirisako T, Takao T, Satomi Y, Shimonishi Y, Ishihara N, Mizushima N, Tanida I, Kominami E, Ohsumi M, Noda T and Ohsumi Y (2000) A ubiquitin-like system mediates protein lipidation. Nature. 408: 488-492
10. Kabeya Y, Mizushima N, Ueno T, Yamamoto A, Kirisako T, Noda T, Kominami E, Ohsumi Y and Yoshimori T (2000) LC3, a mammalian homologue of yeast Apg8p, is localized in autophagosome membranes after processing. EMBO J. 19: 5720-5728
11. Lum J J, Bauer D E, Kong M, Harris M H, Li C, Lindsten T and Thompson C B (2005) Growth factor regulation of autophagy and cell survival in the absence of apoptosis. Cell. 120: 237-248
12. Baird J K (2005) Effectiveness of antimalarial drugs. N Engl J. Med. 352: 1565-1577
13. Lai J H, Ho L J, Lu K C, Chang D M, Shaio M F and Han S H (2001) Western and Chinese antirheumatic drug-induced T cell apoptotic DNA damage uses different caspase cascades and is independent of Fas/Fas ligand interaction. J. Immunol. 166: 6914-6924
14. Issa S N, Ruderman E M (2004) Damage control in rheumatoid arthritis. Hard-hitting, early treatment is crucial to curbing joint destruction. Postgrad Med. 116: 14-14
15. Wozniacka A, McCauliffe D P (2005) Optimal use of antimalarials in treating cutaneous lupus erythematosus. Am J Clin Dermatol. 6: 1-11
16. de Duve C, de Barsy T, Poole B, Trouet A, Tulkens P and Van Hoof F (1974) Commentary. Lysosomotropic agents. Biochem Pharmacol. 23: 2495-2531
17. Seglen P O, Gordon P B (1980) Effects of lysosomotropic monoamines, diamines, amino alcohols, and other amino compounds on protein degradation and protein synthesis in isolated rat hepatocytes. Mol Pharmacol. 18: 468-475
18. Zaidi A U, McDonough J S, Klocke B J, Latham C B, Korsmeyer S J, Flavell R A, Schmidt R E and Roth K A (2001) Chloroquine-induced neuronal cell death is p53 and Bcl-2 family-dependent but caspase-independent. J Neuropathol Exp Neurol. 60: 937-945
19. Boya P, Gonzalez-Polo R A, Poncet D, Andreau K, Vieira H L, Roumier T, Perfettini J L and Kroemer G (2003) Mitochondrial membrane permeabilization is a critical step of lysosome-initiated apoptosis induced by hydroxychloroquine. Oncogene. 22: 3927-3936
20. Boya P, Gonzalez-Polo R A, Casares N, Perfettini J L, Dessen P, Larochette N, Metivier D, Meley D, Souquere S, Yoshimori T, Pierron G, Codogno P and Kroemer G (2005) Inhibition of macroautophagy triggers apoptosis. Mol Cell Biol. 25: 1025-1040
21. Bowman E J, Siebers A and Altendorf K (1988) Bafilomycins: a class of inhibitors of membrane ATPases from microorganisms, animal cells, and plant cells. Proc Natl Acad Sci USA. 85: 7972-7976
22. Drose S, Bindseil K U, Bowman E J, Siebers A, Zeeck A and Altendorf K (1993) Inhibitory effect of modified bafilomycins and concanamycins on P- and V-type adenosinetriphosphatases. Biochemistry. 32: 3902-3906
23. Yoshimori T, Yamamoto A, Moriyama Y, Futai M and Tashiro Y (1991) Bafilomycin A1, a specific inhibitor of vacuolar-type H(+)-ATPase, inhibits acidification and protein degradation in lysosomes of cultured cells. J Biol Chem. 266: 17707-17712
24. Yamamoto A, Tagawa Y, Yoshimori T, Moriyama Y, Masaki R and Tashiro Y (1998) Bafilomycin A1 prevents maturation of autophagic vacuoles by inhibiting fusion between autophagosomes and lysosomes in rat hepatoma cell line, H-4-II-E cells. Cell Struct Funct. 23: 33-42
25. Nishihara T, Akifusa S, Koseki T, Kato S, Muro M and Hanada N (1995) Specific inhibitors of vacuolar type H(+)-ATPases induce apoptotic cell death. Biochem Biophys Res Commun. 212: 255-262
26. Kinoshita K, Waritani T, Noto M, Takizawa K, Minemoto Y, Nishikawa A and Ohkuma S (1996) Bafilomycin A1 induces apoptosis in PC12 cells independently of intracellular pH. FEBS Lett. 398: 61-66
27. Kanzawa T, Kondo Y, Ito H, Kondo S and Germano I (2003) Induction of autophagic cell death in malignant glioma cells by arsenic trioxide. Cancer Res. 63: 2103-2108
28. Kanzawa T, Germano I M, Komata T, Ito H, Kondo Y and Kondo S (2004) Role of autophagy in temozolomide-induced cytotoxicity for malignant glioma cells. Cell Death Differ. 11: 448-457
29. Lee C Y, Baehrecke E H (2001) Steroid regulation of autophagic programmed cell death during development. Development. 128: 1443-1455
30. Martin D N, Baehrecke E H (2004) Caspases function in autophagic programmed cell death in Drosophila. Development. 131: 275-284
31. Gonzalez-Polo R A, Boya P, Pauleau A L, Jalil A, Larochette N, Souquere S, Eskelinen E L, Pierron G, Saftig P and Iroemer G (2005) The apoptosis/autophagy paradox: autophagic vacuolization before apoptotic death. J Cell Sci.
32. Dunn W A, Jr. (1990) Studies on the mechanisms of autophagy: maturation of the autophagic vacuole. J Cell Biol, 110: 1935-1945
33. Punnonen E L, Autio S, Marjomaki V S and Reunanen H (1992) Autophagy, cathepsin L transport, and acidification in cultured rat fibroblasts. J Histochem Cytochem. 40: 1579-1587
34. Jager S, Bucci C, Tanida I, Ueno T, Kominami E, Saftig P and Eskelinen E L (2004) Role for Rab7 in maturation of late autophagic vacuoles. J Cell Sci. 117: 4837-4848
35. Tanida I, Minematsu-lkeguchi N, Ueno T and Kominami E (2005) Lysosomal Turnover, but Not a Cellular Level, of Endogenous LC3 is a Marker for Autophagy. Autophagy. 1: 84-91
36. Wei M C, Zong W X, Cheng E H, Lindsten T, Panoutsakopoulou V, Ross A J, Roth K A, MacGregor G R, Thompson C B and Korsmeyer S J (2001) Proapoptotic BAX and BAK: a requisite gateway to mitochondrial dysfunction and death. Science. 292: 727-730
37. Knudson C M, Tung K S, Tourtellotte W G, Brown G A and Korsmeyer S J (1995) Bax-deficient mice with lymphoid hyperplasia and male germ cell death. Science. 270: 96-99
38. Kuida K, Zheng T S, Na S, Kuan C, Yang D, Karasuyama H, Rakic P and Flavell R A (1996) Decreased apoptosis in the brain and premature lethality in CPP32-deficient mice. Nature. 384: 368-372
39. Shindler K S, Latham C B and Roth K A (1997) Bax deficiency prevents the increased cell death of immature neurons in bcl-x-deficient mice. J. Neurosci. 17: 3112-3119
40. Nowoslawski L, Klocke B J and Roth K A (2005) Molecular regulation of acute ethanol-induced neuron apoptosis. J Neuropathol Exp Neurol. 64: 490-497
41. Wood, D E and Newcomb, E W (2000) Cleavage of Bax enhances its cell death function. J. Exp. Cell Res. 256: 375-382

What is claimed:

1. A method for treating or preventing a disease or condition characterized by neurodegeneration in a subject in need of such treatment or prevention, said method comprising administering to said subject a therapeutically effective amount of at least one macrolide antibiotic or a pharmaceutically acceptable salt, ester, salt of an ester, solvate or prodrug thereof.

2. The method of claim 1 where said therapeutically effective amount is 1 nM or less.

3. The method of claim 1 where said disease or condition is selected from the group consisting of: lysosomal storage disorders (LSDs), Tay-Sach's disease, juvenile neuronal ceroid lipofuscinosis, Niemann-Pick disease, Sandoff's disease, Sanfillippo B syndrome, Alzheimer's disease, frontotemporal dementia, Parkinson's disease, Huntington's disease, FTDP-17, and Lewy body dementia.

4. (canceled)

5. The method of claim 1 where said macrolide antibiotic is selected from the group consisting of: bafilomycin A1, bafilomycin B1, concanamycin and combinations of the foregoing.

6. (canceled)

7. (canceled)

8. (canceled)

9. (canceled)

10. The method of claim 1 where said treatment inhibits, at least in part, neuronal cell death, aberrant neuronal pathology, a death signal generated by lysosomal dysfunction or a death signal generated by autophagic stress.

11. The method of claim 1 where said subject is a human.

12. A method for preventing, at least in part, neurodegeneration in a subject in need of such prevention, said method comprising administering to said subject a therapeutically effective amount of at least one macrolide antibiotic or a pharmaceutically acceptable salt, ester, salt of an ester, solvate or prodrug thereof.

13. The method of claim 12 where said therapeutically effective amount is 1 nM or less.

14. The method of claim 12 where said neurodegeneration is caused by or associated with lysosomal storage disorders (LSDs), Tay-Sach's disease, juvenile neuronal ceroid lipofuscinosis, Niemann-Pick disease, Sandoff's disease, Sanfillippo B syndrome, Alzheimer's disease, frontotemporal dementia, Parkinson's disease, Huntington's disease, FTDP-17, and Lewy body dementia.

15. (canceled)

16. The method of claim 12 where said macrolide antibiotic is selected from the group consisting of: bafilomycin A1, bafilomycin B1, concanamycin and combinations of the foregoing.

17. (canceled)

18. (canceled)

19. (canceled)

20. (canceled)

21. The method of claim 12 where said treatment inhibits, at least in part, neuronal cell death, aberrant neuronal pathology, a death signal generated by lysosomal dysfunction or a death signal generated by autophagic stress.

22. The method of claim 12 where said subject is a human.

23. A method to prevent the detrimental effects of a drug that treats a condition in a subject but which detrimentally impacts, at least in part, a lysosomal pathway or creates autophagic stress in said subject, said method comprising administering a therapeutically effective amount of at least one macrolide antibiotic, or a pharmaceutically acceptable salt, ester, salt of an ester, solvate or prodrug thereof, to said subject in combination with said drug.

24. The method of claim 23 where said subject is a human.

25. The method of claim 23 where said treatment inhibits, at least in part, neuronal cell death, aberrant neuronal pathology, a death signal generated by lysosomal dysfunction or a death signal generated by autophagic stress.

26. (canceled)

27. The method of claim 23 where said macrolide antibiotic is selected from the group consisting of: bafilomycin A1, bafilomycin B1, concanamycin and combinations of the foregoing.

28. (canceled)

29. (canceled)

30. The method of claim 23 where said drug is chloroquine and said condition is malaria, rheumatoid arthritis or an autoimmune disease.

31. (canceled)

32. (canceled)

33. (canceled)

34. (canceled)

35. (canceled)

36. The method of claim 23 where said drug is a fluoroquinolone and said condition is a bone infection, a joint infection, a skin infection, a urinary tract infection, inflammation of the prostate, an ear infections, bronchitis, pneumonia, tuberculosis, a sexually transmitted diseases (STDs), or an infections that affects people with acquired immune deficiency syndrome.

37. The method of claim 36 where said fluoroquinolone is selected from the group consisting of: moxifloxacin, ciprofloxacin, ofloxacin, levofloxacin, lomefloxacin, norfloxacin, enoxacin, gatifloxacin, sparfloxacin and combinations of the foregoing.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. A pharmaceutical composition for the treatment of a disease or condition characterized by neurodegeneration, said pharmaceutical composition comprising a therapeutically effective amount of at least one macrolide antibiotic, or a pharmaceutically acceptable salt, ester, salt of an ester, solvate or prodrug thereof and a pharmaceutically acceptable carrier.

64. The pharmaceutical of claim 63 where said disease or condition is selected from the group consisting of: lysosomal storage disorders (LSDs), Tay-Sach's disease, juvenile neuronal ceroid lipofuscinosis, Niemann-Pick disease, Sandoff's disease, Sanfillippo B syndrome, Alzheimer's disease, frontotemporal dementia, Parkinson's disease, Huntington's disease, FTDP-17, and Lewy body dementia.

65. (canceled)

66. The method of claim 63 where said macrolide antibiotic is selected from the group consisting of: bafilomycin A1, bafilomycin B1, concanamycin and combinations of the foregoing.

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)
